(19) United States
(12) Patent Application Publication
(10) Pub. No.: US 2010/0215584 A1
(43) Pub. Date: Aug. 26, 2010

(54) COMPOSITIONS AND METHODS OF MODULATING THE TASTE AND SMELL RECEPTORS AND SCREENING METHODS THEREFORE

(76) Inventor: James G. Passe, Raleigh, NC (US)

Correspondence Address: PASSE' INTELLECTUAL PROPERTY, LLC, 1717 BRASSFIELD RD., RALEIGH, NC, 27614, US

(21) Appl. No.: 12/095,529

(22) PCT Filed: Nov. 29, 2006
(86) PCT No.: PCT/US2006/061331

§ 371 Date: May 30, 2008

Related U.S. Application Data
(60) Provisional application No. 60/740,974, filed on Nov. 30, 2005.
(60) Provisional application No. 60/741,007, filed on Nov. 30, 2005.

(51) Int. Cl. A61K 49/00; A61K 39/395

(52) U.S. Cl. 424/9.2; 424/130.1

(57) ABSTRACT

The present invention relates to modulation of the perception of either taste or smell through modulation of the activity of GRKs which regulate the desensitization process of the taste and smell receptors. By the process of either inhibiting or enhancing GRK activity at the receptor site a modulation in perception of taste and smell can be achieved. The invention discloses methods of treatment as well as methods of discovering compounds useful in the invention. The invention further discloses methods of treating eating disorders or obesity using compositions of the type disclosed in the invention.

### BACKGROUND OF THE INVENTION

1. Field of the Invention

The present invention relates to methods and compounds useful for modulating perception of the taste and smell receptors. It also relates to screening methods for detecting compositions which modulate the perception of the taste and smell receptors. In particular, the invention relates to compositions and treatments for using compositions which modulate the activity of GRK.

2. Description of the Related Art

The aging of baby boomers combined with the lengthening of the average lifespan have become key drivers of the socio-economic growth and planning in most developed countries. It is estimated that by the year 2030 the over 65 population will reach 65 million. A large proportion of the population older than 65 years has age-related sensory losses that impair overall health, self-sufficiency and quality of life. Decrements in the chemical senses of taste and smell are unfortunate but common aspect of aging Journal of the American Medical Association 278(16), pages 1357-62. In addition to the 3.0 million patients with essentially complete loss of taste and smell, 40% of the population over the age of 60 has a significant smell deficit and half of individuals 65 to 80 years old are experiencing noticeable loss of smell. The National Institutes of Health (NIH) has decided to raise awareness on the decline of smell and taste with age or illness by launching, on Aug. 8, 2005, a whole section dedicated to the problem, an area of their Senior Health website that reflects the emergence of a drive to bring solutions to these conditions for which there are no available treatments today.

In addition to these problems, as flavor and fragrance companies continue to attempt to provide products which improve the presentation of foods perfumes and the like there is a constant and continuing need to provide compositions which modulate the taste and smell of food products, perfumes and such products to improve their palatability and acceptability in the market. These companies are constantly searching for new compositions which can act to improve flavors much as monosodium glutamate as done in the past. Products such as soy containing foods, and other legumes and organic products often have less than acceptable flavors and fragrances and could be greatly improved with a composition that modulates up or down the perception of the taste and smell receptors.

Odors and tastes are perceived jointly as flavors via combinations of smell receptors located in the nose and taste receptors found on the tongue. The smell receptors and the sweet, bitter and umami taste receptors are part of the large G protein-coupled receptor (GPCR) family that transduces a wide variety of extracellular ligands into intracellular signaling events. Smell and taste receptors are closely linked as is the entire GPCR family of receptors. The taste receptors of the tongue identify taste; the smell receptors in the nose identify smell. Both of these receptors are part of the family of GPCRs that are involved in perception (as are eye receptors). The remaining GPCRs are involved in metabolic events such as pain perception and regulation, melanogenisis and heart rate. The NIH refers to taste and smell disorders as well as those in hearing and sight as “communication disorders”.

Both taste and smell sensations are communicated to the brain, which integrates the information so that flavors and fragrances can be recognized and appreciated. Some simple tastes—such as salty, bitter, sweet, and sour—can be recognized without the sense of smell. However, more complex flavors (raspberry, for example) require both taste and smell sensations to be recognized.

Many people mistakenly believe that they have a problem with taste, when they are actually experiencing a problem with smell. It is common for people who lose their sense of smell to say that food has lost its taste. Decreased perception of odors and tastes may present some serious medical problems for many older Americans. For instance, loss of smell can lead to: nutritional deficiencies, decreased sex drive, depression, accidental food poisonings and deaths.

Loss of taste perception or food that does not taste good can alter food choices and patterns of consumption producing: weight loss, malnutrition, impaired immunity, accidental food poisonings and death

Anyone experiencing loss of taste and smell functions, regardless of age, are gradually deprived of joys that constitute the basis of one's wellbeing such as love for fine foods or wines or scents of nature. This contributes to further diminishing and isolating persons mentally and socially, by shutting down their access to a variety of pleasurable events and making it impossible to share these positive experiences with others.

The consumption of excessive amounts of salt in processed foods and sugar in soft drinks further diminishes the ability to discriminate nuances of flavor, contributing to taste and smell problems in pre-senior populations. To compensate for that loss of taste and smell, many tend to supplement their foods with strong spices and additives, which may lead to a further decline of those senses.

As a result of recent advances in genomics over the past decade, many of the proteins that mediate biological processes have been identified. This success brings us to a stage where once these proteins are discovered as well as how they work in a biological system, it is possible that mediators of the biological processes could be identified which might modify positively or negatively such processes. However, absent the discovery of the proteins and that they actually modulate such processes such mediators can not be discovered.

The taste and smell G protein-coupled receptors (GPCRs) are cell surface receptors that perceive and signal the presence of tastants and odorants to the brain. It is known that certain intracellular proteins, such as GPCR kinases (GRK's), regulate the internalization of all of the several hundred extracellular GPCRs. GPCRs form a large physiologically important protein family, whose members are gatekeepers to multiple biochemical pathways. GRKs are kinases that modify the internalization of GPCRs by adding phosphate groups to determinants found throughout the intracellular portion of the receptor. GRKs 2, 3, 5 and 6 are the isoforms most strongly associated with smell and taste GPCRs although it appears that all the GRK family might have some function in taste and smell.

While modulation of GRK associated signal transduction has been shown to be useful in a variety of metabolic disease states such as diabetes, hypertension, obesity, dyslipidemia, congestive heart disease, arteriosclerosis, cholesterolinemia coagulation disorders and syndrome X, (published patent application US 2003/004103) to date there is currently no treatment for GPCR based perception disease, such as the age related loss of the total number of GPCR taste and smell receptors, which can fully or partially modulate smell or taste sensations due to such receptor loss. There is also, no test for or compositions for the modulation of the taste and smell GPCRs that would change or modulate the taste perception of flavors and fragrances.

Modulators and methods of finding modulators that either increase or decrease the GRKs activity are taught for example in U.S. Pat. No. 6,833,436 and US 2002/0028772 in that modulators of GRK 2 and GRK 3 are disclosed and that modulate the other GRK's. Disclosed are peptides, low molecular weight organic molecules, anti sense compounds, antibodies, and the like which can achieve such modulation. Only an affect on metabolic receptors is taught. Diabetes, obesity, Syndrome X, heat disease, atherosclerosis and type II DM. It is taught that the primary method of administration is systemic and that multiple systems will be affected simultaneously. Local administration is also taught for enhanced metabolic melanogenisis via down regulation of MSH. The specific example treats melanoma cells in vitro and no examples of mammalian treatment in vivo are disclosed or discussed. The application also does not disclose the treatment of taste or smell perception related disease or taste and smell modulation nor the application either directly or indirectly to any taste or smell receptor of a modulator. It further does not teach that they are possible food additives or that modulation of taste is even possible. It also does not teach the utility of use of such inhibitors for conditions of reduced numbers of receptors such as is the condition for age related loss of taste and smell receptors.

### SUMMARY OF THE INVENTION

It is discovered that the application of a GRK inhibitor directly to the GPCR taste receptors on the tongue or the smell receptors of the nose in a subject will modulate the perception of taste and smell. This is surprisingly accomplished without significant modulation of any metabolism processes controlled by the GRK or any other significant systemic affect. Accordingly, one aspect of the invention is a method for modulation of the perception of GPCR taste or smell in a subject comprising administering to the subject an effective amount of a formulation which contains a composition which inhibits the activity of a GRK protein directly to the tongue or the nose sufficient to effect a perceptible modulation the ability to detect taste or smells.

In another aspect of the invention there is disclosed, a formulation for modulating the perception of taste or smell comprising a composition which inhibits the activity of GRK formulated for administration directly to the GPCR taste receptors on the tongue or the smell receptors in the nose.

In another aspect of the invention there is disclosed a food or an odorant to which has been added a GRK modulator sufficient to modulate a subjects perception of a taste or smell of the food or odorant when consumed.

Still another aspect of the invention comprises a method of reducing the amount of food consumed in a subject comprising administering a GRK inhibitor which enhances unpleasant taste or smell.

In yet another aspect of the invention there is disclosed a method of screening for compounds capable of modulating taste or smell comprising:
a. selecting a composition which inhibits the activity of GRK;
b. administering the composition to a test subject such that GRK in the vicinity of the taste or smell receptors is inhibited;
c. determining if the administration of the composition modulates the subject's perception of smell or taste; and
d. selecting compounds which exhibit modulation of smell or taste.

In yet another aspect of the invention there is disclosed various compounds, formulations and compositions for treatment of age related loss of smell and taste and for modulation of flavors and fragrances.

In yet another aspect of the invention a food or odorant has a GRK inhibitor added sufficient in concentration to modulate the perception of smell or taste.

It is an aspect of this invention to be able to treat the age related loss of the taste and smell receptors in a subject by administering a GRK inhibitor directly to the site of those receptors in order to modulate the perception of taste and smell.

In another aspect of the invention there is a method of treating obesity by administering to a subject in need of treatment, a GRK inhibitor wherein such inhibitor enhances unpleasant tastes such as the bitter taste receptors.

In yet another embodiment of the invention a method for inhibiting the feeding of insects is disclosed comprising administering to an insect an amount of a GRK inhibitor sufficient to modulate the taste of insects such that undesirable tastes increased to an extent that the insect reduces or eliminates feeding.

### DETAILED DESCRIPTION OF THE INVENTION

Both the smell and many of the taste receptors of mammals, insects and the like are GPCRs. GPCR kinases (GRKs) play a role in phosphoralyating and regulating the desensitization of the GPCRs. It is known that all GRKs and especially GRK 2, 3, 5 and 6 are present in the tongue and nasal cavity and it has been postulated that they are involved in the desensitization of the taste and smell receptors. It is now disclosed that where a GRK inhibitor is applied directly to the locus of a smell or taste receptor in a subject such as a mammal or insect, for example, those remaining in age related loss of smell or taste, that the receptors will signal longer or stronger and the subject will experience modulated sense of taste or smell. This is especially true when the subject has a reduced number of receptors for example a person with a reduced number of receptors due to age related loss of the total number of receptors.

It is also disclosed that when a GRK modulator (one that increases or decreases GRK) is applied directly to the locus of the GPCR smell or taste receptor, for example, a food ingredient, flavor or fragrance or the like that the receptors modulate the taste or smell perceived by the subject.

GRKs are a family of serine/threonine kinases that induce receptor desensitization by the phosphorylation of agonist-occupied or -activated receptors. GRKs transduce the binding of extracellular ligands into intracellular signaling events. To date, seven members of the GRK family have been identified. Common features of these kinases include a centrally localized catalytic domain of approximately 240 amino acids, which shares significant sequence identity between family members, an N-terminal domain of 161-197 amino acids, and a variable length C-terminal domain.

This invention relates to the 7 member GRK family of kinases. Both the structure and activity of the GRKs has been well studied. Especially the following GRKs have been implicated as desensitizing the taste and smell receptors.

### GRK2

GRK2 is responsible for the desensitization of adrenaline receptors in the heart and is essential for proper heart development. However, abnormal levels of GRK2 have also been linked to congestive heart failure, high blood pressure, and opiate addiction. GRK2 is a cytoplasmic protein that targets activated GPCRs via the interaction of its pleckstrin homology (PH) domain with the heterotrimeric G-protein subunits β and γ (Gβγ). Free Gβγ subunits are released from Gα subunits only after GPCR activation. ALS NEWS Vol 237, Jan. 28, 2004. In Chemical Senses 2005 30(4):281-290 it is taught that GRK 2 is present in taste bud cells and that that three GRKs (GRK2, GRK3 and GRK5) are differentially distributed in the circumvallate papilla.

### GRK3

GRK3 as used herein is the GRK kinase associated with desensitization of various GPCRs. It is part of the subfamily of GPCR kinases known as β-adrenergic receptor kinases. Its sequence is well known and its effect well studied. see e.g. J. Biol chem 1997 October 10:272 (41):25425-25428. GRK3 appears to be the prime kinase associated with the phosphorylization of the olfactory (smell) receptor and is known to when blocked lead to loss of the odorant receptor desensitization. It does appear to be a minor component of the kinases associated with the taste receptors. It is taught herein as part of this invention that application of an inhibitor of GRK3 directly to the site of a smell or taste receptor will block desensitization of the receptor leading to a longer signaling time and therefore an increased perception of the sense of taste or smell. When stimulated by a tastant or odorant. Normally, the systemic inhibition of GRK3 in the entire mammal can lead to disruptive problems such as reports that inhibition of GRK3 leads to bipolar symptoms interference with opiate tolerance and a host of other symptoms and problems.

### GRK 5

GRK5 as used herein is the GRK also associated with various receptors' desensitization profile. It is part of the subfamily of GPCR kinases known as the GRK4 kinases. Its sequence is well known and its effect well studied. See e.g. U.S. Pat. No. 6,255,069. GRK5 is a protein of approximately 67.7 kDa (see Kunapali and Benovic (1993) P.N.A.S. 90:5588-5592) and was identified by its homology with other members of the GRK family. It is expressed in a number of different tissues, including heart, placenta and lung. Autophosphorylation of GRK5 appears to activate the kinase (Pronin and Benovic (1997) P.N.A.S. 272:3806-3812). GRK5 is also phosphorylated by PKC, where the major sites of PKC phosphorylation are localized within the C-terminal 26 amino acids. PKC phosphorylation significantly inhibits GRK5 activity. GRK5 over expression inhibits thrombin-activated signaling and expression of a dominant negative GRK5 mutant prolongs thrombin-activated Ca++ signaling in endothelial cells.

### GRK 6

It is also postulated that GRK 6 is present in taste bud cells. In AMJ Physiol Cell Physiol 289: C483-C492 it is taught that it appears that GRK 6 is involved in the phosphorilization of bitter GPCR receptors and also interacts intracellularly with the receptors downstream shutoff components to inhibit signal termination

As used herein “effective amount” is meant a sufficient amount of a GRK modulator (one that inhibits or enhances GRK) that when applied directly to the tongue or intranasally (or directly to those GPCR taste or smell receptors wherever present) in a subject having GPCR taste or smell receptors such as a mammal or insect, that is sufficient to elicit a noticeable modulation in the ability to detect or perceive smells or taste or the duration of detection of tastants or odorants or to elicit a change or modulation in the perceived tastant or odorant. It is in an embodiment of the invention that modulation occurs without any significant changes to the systemic metabolic effects to which a GRK modulator could have on the subject. In one embodiment the effective amount is sufficient to selectively modulate a desired taste or smell over other tastes and smells, for example the enhancement of bitter tastes which would be useful in treating obesity or in inhibiting the feeding by insects or the inhibition of bitter tastes which would improve the taste of a particular food.

As used herein “mammal” is meant preferably a human but also other mammals that may benefit from improved taste or smell such as improved feeding or longer feeding and the like in veterinary animals such as cats and dogs, farm animals such as cows, chickens, pigs and laboratory animals such as rats and mice. As used herein “subject” is any organism with taste or smell GPCRs.

As used herein “modulating” refers to mean altering or changing the perception of a tastant or odorant by a mammal compared to a base line perception for the individual mammal. It can be measured by improved detection of a taste or smell or by improved feeding such as longer feeding more vigorous feeding or the like. It can also include improved taste or smell perception or it can include making tastes or smells perceived as worse or unpleasant.

As used herein “perceptible” means a measurable change such that the subject exhibits a desirable improvement in the ability to perceive tastes and smells to a higher degree than without the treatment or exhibits improved feeding characteristics or more enthusiastic feeding characteristics. It may be selective for a given taste or enhance or inhibit the ability to taste or smell non-selectively. It may modulate to a previously unknown amount as well but in any event change the current perception of the taste or smell.

As used herein “minimal systemic absorption” means that a formulation for application to the tongue or nose or otherwise to the GPCR taste or smell receptor is so formulated or the ingredient so chosen so as to minimize the GRK modulation from substantially having a systemic effect.

As used herein “threshold of perception” is meant that the ability of a subject to detect changes in the perceived taste or smell of a tastant or odorant. This would be the case either before or after administration of a composition of the present invention.

There are many compositions that are already known to modulate the activity of GRK. They include low molecular weight organic molecules, anti-sense nucleic acids, negative dominant GRK genes, immunoreactive antibodies and peptides. In addition, it is well within the skill in the art to test compositions for their ability to inhibit the activity of a selected GRK on the phosphorylization of GPCRs. For example, the art teaches incubating cells with a test substance to produce a test mixture for assessment on the activity of the given GRK. The activity is assessed based on the activity of a given receptor and compared with a suitable control for example the activity of the same cells incubated under the same conditions in the absence of the test substance. Typical tests involve the measurement of cellular activity which is known to be controlled by the GRK. The easiest to measure are the metabolic activities of the cell and assessment is made by a quantitative comparison.

Because the methods of the invention relate to perception detection (i.e. smell and taste much like vision which is also controlled by GPCR perception) and because various modulators may have a varying effect on the GPCR activity intensity and result depending on the mode of administration and the particular receptor detected an additional test needs to be performed in order to determine if the modulator can be used to treat a condition or achieve a result related to taste and smell as controlled by the taste and odor receptors. Once the composition is known to modulate the desired GRK the composition is administered to a test mammal in vivo such that an effective dose is applied to the nasal receptors or tongue receptors. The mammal is then tested to determine if there is a change in the perception of taste or smell. This can be done in a human for example by having a human test subject attempt to identify a threshold amount of a tastant or odorant both before and after administration of the test composition. In the case of a test animal the animal can be looked at in terms of improved feeding activity in volume or rapidity compared to feeding without the composition. One skilled in the art could easily determine other tests based on the disclosure provided herein.

Formulation of the GRK modulator for use in the present invention is made with attention to the mode of delivery. Since administration is intended to be by application to the tongue or nasal mucosa i.e. intraoral or intranasal and in one embodiment without substantial systemic absorption certain parameters would be desirable. The inhibitor would be formulated with those additional ingredients that would make the formulation deliverable as desired.

Acceptable excipients could be added means an excipient that is useful in preparing a composition that is generally safe, non-toxic and neither biologically nor otherwise undesirable, and includes excipients that are acceptable for veterinary use as well as human use. A “pharmaceutically acceptable excipient” as used in the specification and claims includes both one and more than one such excipient.

“Acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

The composition may be formulated into a preparation using a filler, a thickening agent, a binder, a humectant, a disintegrator, a diluent such as a surfactant, or an excipient. Examples of solid preparations for oral administration to the tongue area include tablets, pills, powders, gels, ointments and the like. Such solid preparations may contain one or more excipients selected from starch, calcium carbonate, sucrose or lactose, and gelatin. In addition to the simple excipient, the solid preparations may also contain a lubricant such as magnesium stearate or talc. Examples of liquid preparations for oral administration include suspensions, liquid solutions such as elixirs, emulsions and syrups. The liquid preparations may contain a simple diluent such as water or liquid paraffin, and various excipients, which are exemplified by humectants, sweetening agents, aromatic agents and preservatives.

A nasal preparation comprised of the composition described above can take a variety of forms for administration in nasal drops, nasal spray, gel, ointment, cream, powder or suspension, using a dispenser or other device as needed. A variety of dispensers and delivery vehicles are known in the art, including single-dose ampoules, atomizers, nebulae's, pumps, nasal pads, nasal sponges, nasal capsules, gel strips and the like.

More generally, the preparation can take a solid, semi-solid, or liquid form. In the case of a solid form, the components may be mixed together by blending, tumble mixing, freeze-drying, solvent evaporation, co-grinding, spray-drying, and other techniques known in the art. Such solid state preparations preferably provide a dry, powdery composition with particles in the range of between about 20 to about 500 microns, more preferably from 50 to 250 microns, for administration intranasally.

A semi-solid preparation suitable for intranasal administration can take the form of an aqueous or oil-based gel or ointment. For example, the components described above can be mixed with microspheres of starch, gelatin, collagen, dextran, polylactide, polyglycolide or other similar materials that are capable of forming hydrophilic gels. The microspheres can be loaded with drug, and upon administration form a gel that adheres to the nasal mucosa and might also be used to apply intraorally.

Useful surface stabilizers physically adhere to the surface of the active substance but do not chemically bond to or interact with the active substance. The surface stabilizer is adsorbed on the surface of the active substance while the individually adsorbed molecules of the surface stabilizer are essentially free of intermolecular cross-linkages. Accordingly, one or more surface stabilizers can be employed in a composition and method of the present invention. Examples of suitable surface stabilizers include, but are not limited to, various polymers, low molecular weight oligomers, natural products, and nonionic and ionic surfactants. Examples of surface stabilizers include gelatin, glycerine, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters [e.g., a commercially available Tween®: Tween 20®, Tween 80®, (trade names of ICI Specialty Chemicals) and the like]; a synthetic glycol polymer (such as propylene glycol, polypropylene glycol, polyethylene glycol, polyvinylpyrrolidone, polyvinylalcohol, polyoxyethylene copolymers, polyoxypropylene copolymers, polyethyleneoxide and the like: e.g., Carbowax 3550® or Carbowax 934® (trade names of Union Carbide)), polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethyl-cellulose phthalate, noncrystalline cellulose, magnesium aluminum silicate, triethanolamine, polyvinyl alcohol (PVA), polyvinylpyrrolidone (PVP), 4-(1,1,3,3-tetramethylbutyl)-phenol polymer with ethylene oxide and formaldehyde (also known as tyloxapol, superione, and triton), poloxamers (a block copolymer of ethylene oxide and propylene oxide) (e.g., Pluronics F68® and F108®); poloxamines (a tetrafunctional block copolymer derived from sequential addition of propylene oxide and ethylene oxide to ethylenediamine) (e.g., Tetronic 908®, also known as Poloxamine 908® or Tetronic 1508®, also known as T-1508, all trade names of the BASF Wyandotte Corporation); dialkylesters of sodium sulfosuccinic acid (e.g., a dioctyl ester of sodium sulfosuccinic acid) (Aerosol OT®, a trade name of American Cyanamid); a sodium lauryl sulfate (e.g., Duponol® P, a trade name of DuPont); an alkyl aryl polyether sulfonate (e.g., Tritons® X-200, a trade name of Rohm and Haas); a mixture of sucrose stearate and sucrose distearate (e.g., Crodestas® F-110 or Crodestas® SL-40, trade names of Croda Inc.) and the like. Most of these surface stabilizers are known pharmaceutical excipients and are described in detail in the Handbook of pharmaceutical Excipients, published jointly by the American Pharmaceutical Association and The Pharmaceutical Society of Great Britain.

Thickening agents act to impart viscosity to the aqueous medium. Examples of thickening agents include, but are not limited to, natural gums (such as acacia, xanthan gum and the like), a high molecular weight cross-linked acrylic acid carbomer [such as Carbopol® 980, Carbopol® 974P (Carbomer 934P), Carbopol® 940 (all trade names of B.F. Goodrich & Co.)] and the like or mixtures thereof.

Lubricating agents act on the ability of the active substance and resin powders to flow. Examples of lubricants include, but are not limited to, colloidal silicon dioxide (such as Aerosil® 200), talc, stearic acid, magnesium stearate, calcium stearate or silica gel.

Suspending agents act on the ability of the resinate to remain distributed in a suspension and thus maintain content uniformity of the active substance in suspension. Examples of suspending agents include, but are not limited to, propylene glycol, polyethylene glycol, glycerin and the like or mixtures thereof.

Neutralizing agents in the context of the present invention shift the equilibrium concentration of a solubilized weakly basic active substance and drive the active substance to favor complexing with a weakly acidic ion-exchange resin. The equilibrium concentration is shifted since a neutralizing agent is used to remove excess solubilized hydrogen ions present in the suspension as a result of using various acidic components (such as an acidic resin, an acidic carrier material, an acidic thickening agent and the like). Examples of neutralizing agents include, but are not limited to, sodium hydroxide.

Sweetening agents, flavoring agents and mixtures thereof used in the present invention are selected from those which are pharmaceutically acceptable, compatible with the attributes of an oral dosage formulation and adequately mask a slight acidic taste to below the taste threshold. Examples of sweetening agents include any natural or artificial sweetener (such as glucose, dextrose or fructose and the like or mixtures thereof, when not used as a carrier; saccharin and its various salts, cyclamate, aspartame, acesulfame-K and its sodium and calcium salts and the like or mixtures thereof; sucrose or sucralose; sugar alcohols such as sorbitol, mannitol, xylitol and the like or mixtures thereof) and the like or mixtures thereof.

Examples of flavoring agents include any natural or synthetic flavoring liquid (such as volatile oils, synthetic flavor oils, flavoring aromatics, oils, liquids, oleoresins and extracts derived from plants, leaves, flowers, fruits, stems and combinations thereof, including, but not limited to, spearmint, peppermint, lemon, orange, grape, lime or grapefruit citric oils or apple, pear, peach, grape, strawberry, raspberry, cherry, plum, pineapple, apricot, or other mint or fruit flavor essences), an aldehyde or ester (such as benzaldehyde (cherry, almond), citral, a-citral (lemon, lime), neral, beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanal (green fruit), 2-dodedenal (citrus, mandarin) and the like or mixtures thereof.

Examples of coloring agents include any pharmaceutically acceptable natural or synthetic dyes (such as Red 30 ferric oxide and the like) and the like or mixtures thereof.

Examples of anti-foaming agents include, but are not limited to, simethicone and the like or mixtures thereof.

Examples of preservatives include, but are not limited to, potassium sorbate, methylparaben, propylparaben, benzoic acid and its salts, other esters of parahydroxybenzoic acid (such as butylparaben, alcohols such as ethyl or benzyl alcohol), phenolic compounds (such as phenol) or quarternary compounds (such as benzalkonium chloride).

The amount of modulator administered to a subject either intraorally or intranasally will depend on a number of factors. The severity of the loss of taste or smell and the characteristics of the particular subject being treated such as general health, body weight, age, sex and general tolerance to drugs. In general though, the amount and concentration will be determined based on the activity of the modulator and the route of administration. Since the modulator is designed to work directly on the receptor, the amount of modulator and its activity can be much less that if it were being given for systemic uptake. Accordingly, the need for chemistry optimization may in some cases be avoided entirely and even natural compositions or nutraceutical type preparations may be used at lower dosages. The exact amount can easily be determined by one skilled in the art by the screening method testing described herein, that is by direct testing on the mammal or subject of interest.

Since it is an embodiment of this invention to prevent as much as is reasonable, systemic absorption, the selection of the formulation will be with that object in mind. Intraoral administration can be by a number of methods. Dissolving tablets, gel strips, ointments, creams, sprays and the like can be used. For intranasal administration sprays, ointments, creams, inhalers and the like can be used. In general the inhibitor would be used prior to consumption of food or drink or prior to an activity which requires taste or smell such as a dangerous environment which requires the sense of smell (e.g. chemical or oil manufacturing plant). Since the administration is direct to the receptor locus, the onset of activity will be relatively quickly, often half hour or less and in some cases less that ten to fifteen minutes. In some cases the onset of activity may be relatively instantaneous. The time that the modulator will last will be based on a number of factors including the particular modulator, the physical condition of the subject, the formulation used to deliver the modulator and the like.

The invention is illustrated further by the following examples which are not intended to be limiting.

### Example 1

### Testing Humans for Treatment

A formulation comprising a composition determined to be a GRK5 inhibitor is formulated for oral administration directly to the tongue. The composition is formulated in a series of concentrations to determine the optimum activity if any of the composition in treating loss of taste receptors in the test subject. A base line threshold determination is made by allowing a series of foods with differing concentrations of a single tastant to be tasted and a determination of which are perceived and which are not. Following that, the test composition is applied in the same manner and the inhibitors effectiveness and optimized concentration is determined.

### Example 2

### Testing of an Animal Subject

A GRK 2, 3 5 or 6 inhibitor at various concentrations is applied to the tongue of a test rat (or mouse) or incorporated into the food of the test animal. The feeding activity of the animal is measured before and after the treatment for determination of the optimized formulation and concentration.

### Example 3

### Testing for Smell Improvements

A GRK3 inhibitor is incorporated into a formulation suitable for administration either to the nasal cavity. A base line is measured as in Examples 1 and 2 and administered to humans and animals intranasally to determine the effectiveness in perception of test odors in comparison to before administration of the inhibitor. Compositions are selected based on optimized dosage, effect lack of systemic absorption and the like.

### Example 4

### Formulation for Oral Administration

A GRK2 inhibitor is formulated first into an oral solution for application to the tongue. A second inhibitor is formulated into a gel strip for dissolution on the tongue.

### Example 5

### Formulation for Nasal Administration

A GRK3 inhibitor is formulated at an effective therapeutic amount into a nasal spray formulation so formulated to minimize systemic absorption of the inhibitor.

### Example 6

Either a GRK2, 3, 5 or 6 modulator is mixed into a food or drink intended for conception by a subject. The amount of the added modulator is determined such that improved feeding is accomplished.

### Example 7

A GRK modulator is fed to a test subject, either a mammal or an insect and decreased feeding is then determined to be a positive result for either an anti obesity composition or an insect feeding inhibitor.

While the invention described herein is described with references to certain embodiments, it is clearly understood that those skilled in the are will be able to make changes in form and details and be able to substitute inhibitors, methods of administration and the like without deviating from the scope of the invention as taught and claimed herein.

What is claimed is:

1. A method for modulation of the perception of taste or smell in a subject comprising administering to the subject an effective amount of a formulation which contains a composition which inhibits the activity of a GRK protein directly to the tongue or the nose sufficient to effect a perceptible modulation of taste or smell.

2. A method according to claim 1 wherein the GRK is selected from the group consisting of GRK2, GRK3, GRK5 or GRK6.

3. A method according to claim 1 wherein the composition is applied to the tongue.

4. A method according to claim 1 wherein the composition is applied to the nasal cavity.

5. A formulation for modulating the perception of taste or smell comprising a composition which inhibits the activity of GRK formulated for administration directly to the taste receptors on the tongue or the smell receptors in the nose.

6. A formulation according to claim 5 wherein the formulation is formulated for application intranasally.

7. A formulation according to claim 5 wherein the formulation is formulated for application to the tongue.

8. A formulation according to claim 5 which has been formulated to minimize systemic absorption of the composition.

9. A food or an odorant to which has been added a GRK modulator sufficient to modulate a subject's perception of a taste or smell of the food or odorant when consumed.

10. An odorant according to claim 9 which is a perfume.

11. A food according to claim 9 which is an animal feed.

12. A food according to claim 9 which is intended for human consumption.

13. an odorant according to claim 9 which is intended for human perception.

14. A method of reducing the amount of food consumed in a subject comprising administering a GRK modulator which enhances unpleasant taste or smell.

15. A method according to claim 14 wherein the subject is a human bitter taste in enhanced.

16. A method according to claim 14 wherein the subject is an insect.

17. A method of screening for compounds capable of modulating taste or smell comprising:
a) selecting a composition which inhibits the activity of GRK;
b) administering the composition to a test subject such that GRK in the vicinity of the taste or smell receptors is inhibited;
c) determining if the administration of the composition modulates the subject's perception of smell or taste; and
d) selecting compounds which exhibit modulation of smell or taste.

18. A method according to claim 17 wherein the composition is administered directly to the smell receptors in the nose.

19. A method according to claim 17 wherein the composition is administered directly to the taste receptors on the tongue.

20. A method according to claim 17 which further comprises the steps of:
a) determining if the level of systemic absorption of the composition; and
b) selecting those compositions which modulate taste or smell and which do not exhibit significant systemic absorption.
